(19) United States
(12) Patent Application Publication
(10) Pub. No.: US 2009/0156992 A1
(43) Pub. Date: Jun. 18, 2009

(54) NEEDLE-FREE INJECTION DEVICE AND PRIMING SYSTEM

(75) Inventor: Sergio Landau, Laguna Niguel, CA (US)

Correspondence Address: KOLISCH HARTWELL, P.C., 200 PACIFIC BUILDING, 520 SW YAMHILL STREET, PORTLAND, OR, 97204, US

(73) Assignee: BIOJECT, INC., Tualatin, OR (US)

(21) Appl. No.: 12/390,300

(22) Filed: Feb. 20, 2009

Related U.S. Application Data
(62) Division of application No. 11/627,298, filed on Jan. 25, 2007.
(60) Provisional application No. 60/762,567, filed on Jan. 27, 2006.

(51) Int. Cl. A61M 5/30

(52) U.S. Cl. 604/68

(57) ABSTRACT

Needle-free injection devices having a delivery system to effect an injection and a body configured to house the delivery system. The delivery system includes an injectate assembly adapted to house a volume of liquid and a drive assembly adapted to expel the volume of liquid from the injectate assembly. In some embodiments, the drive assembly includes a pair of parallel springs. The device further includes a priming system adapted to prepare the device for delivery of an injection. The priming system includes a force-preparation assembly adapted to selectively arrange the drive assembly to provide the drive force to the injectate assembly. In some embodiments, the priming system includes a locking assembly adapted to releasably retain the injectate assembly relative to the body. In some embodiments, the priming system includes a dosing assembly adapted to selectively draw a volume of liquid into the injectate assembly.

### CROSS-REFERENCE TO RELATED APPLICATIONS

This application is a divisional of U.S. patent application Ser. No. 11/627,298, filed Jan. 25, 2007 and entitled “NEEDLE-FREE INJECTION DEVICE AND PRIMING SYSTEM,” which application is based upon and claims priority under 35 U.S.C. 119(e) to U.S. Provisional Patent Application No. 60/762,567, filed Jan. 27, 2006 and entitled “NEEDLE-FREE SPRING-LOADED INJECTION SYSTEM,” the disclosure of which is incorporated herein by reference.

### BACKGROUND

Needle-free injection systems provide an alternative to standard fluid delivery systems, which generally use a needle adapted to penetrate the outer surface of a target. Typically, needle-free injection systems are designed to eject the fluid from a fluid chamber with sufficient pressure to allow the fluid to penetrate the target to the desired degree. For example, common applications for needle-free injection systems include delivering intradermal, subcutaneous and intramuscular injections into or through a recipient's skin. For each of these applications, the fluid must be ejected from the system with sufficient pressure to allow the fluid to penetrate the tough exterior dermal layers of the recipient's skin.

One method for generating sufficient pressure is to use a spring powered device, such as those described in U.S. Pat. Nos. 4,592,742, 5,062,830, 5,782,802, and 6,506,177 and U.S. Published Patent Application No. 2005/0119608 A1, the disclosures of which are incorporated herein by reference. These devices include a single force-generating spring and an injection ram arranged linearly along the same axis.

### SUMMARY

The present disclosure is directed to needle-free injection devices having a delivery system to effect an injection from a body of the device. The delivery system includes an injectate assembly that houses a volume of liquid and a drive assembly that expels the liquid from the injectate assembly. The drive assembly may include a pair of parallel springs configured to simultaneously deliver an operative force to expel the liquid from the injectate assembly. The delivery system may include a transmission assembly adapted to couple the injectate assembly and the drive assembly. The injection devices further include a priming system to prepare the device for delivery of an injection. The priming system may include a force-preparation assembly to selectively compress the pair of springs. The priming system may include a locking assembly adapted to releasably retain the injectate assembly relative to the body.

The advantages of the disclosed needle-free injection system may be understood more readily after a consideration of the drawings and the Detailed Description.

### BRIEF DESCRIPTION OF THE DRAWINGS

FIG. 1 is a perspective view of an exemplary device including a body configured to house a priming system and a delivery system.

FIG. 2 is a schematic diagram of suitable priming and delivery systems for a needle-free injection device, such as the device of FIG. 1. The priming system includes a force-preparation assembly and may include a locking assembly and a dosing assembly. The delivery system includes an injectate assembly, a drive assembly, a trigger assembly, and may include a transmission assembly.

FIG. 3 is a perspective view of a portion of the device of FIG. 1 illustrating insertion of an exemplary injectate assembly into an exemplary locking assembly while the locking assembly is in an open position.

FIG. 4 is a perspective view of the device of FIG. 3 illustrating the locking assembly in a closed position.

FIG. 5 is a perspective view of the device of FIG. 1 with a portion of the housing removed and showing an exemplary drive assembly.

FIG. 6 is an exploded view of the exemplary device of FIGS. 1 and 2.

FIG. 7 is a side elevation cross-sectional view of the device of FIG. 6 in a stored configuration in which the drive assembly of the delivery system is not operatively coupled to the injectate assembly.

FIG. 8 is a side elevation cross-sectional view of the device of FIG. 7 in a primed configuration in which the drive assembly operatively couples with the injectate assembly via the transmission assembly and the drive assembly is prepared to provide a drive force to the injectate assembly.

FIG. 9 is a side elevation cross-sectional view of the device of FIG. 8 in a dosed configuration in which the injectate assembly is filled.

FIG. 10 is a side elevation cross-sectional view of the device of FIG. 9 upon actuation of the trigger assembly to alter the device to a fired configuration in which the drive assembly transmits a driving force to the injectate assembly.

FIG. 11 is a side elevation cross-sectional view of the device of FIG. 10 in a fired configuration upon completion of delivery of the injection.

FIG. 12 is a top elevation cross-sectional view of the exemplary device of FIG. 7.

FIG. 13 is a perspective view of another exemplary locking assembly illustrating movement of the locking assembly between open and closed positions.

FIG. 14 is a top elevation cross-sectional view of the locking assembly of FIG. 13.

FIG. 15 is a side elevation cross-sectional view of the locking assembly of FIG. 13 illustrating the locking assembly without an injectate assembly.

FIG. 16 is a side elevation cross-sectional view of the locking assembly of FIG. 13 with an injectate assembly inserted.

FIG. 17 is a perspective view of an electric winder suitable for use with the device of FIGS. 1-16.

### DETAILED DESCRIPTION

FIGS. 1-16 illustrate exemplary systems and components for a needle-free injection device 10. Although the disclosed device is intended to be reusable, various aspects of the device may be incorporated into single-use, disposable devices.

Device 10 includes a body 12 to house various systems used to effect an injection. As illustrated in FIG. 1, body 12 includes a front housing 14, a central housing 16, and a rear housing 18. The housing sections may include a variety of apertures, such as opening 20 in the front housing, opening 22 in the central housing, and opening 24 in the rear housing, to enable access to and/or coupling of device components. The housings may be configured to move relative to one another to actuate the various systems. For example, one or more of the housings may be rotatable about an axis 30 to actuate various systems of the device. Body 12 is typically sized and shaped to be comfortably held in a user's hand and may take any suitable configuration. Body 12 may be formed from injection-molded plastic, though various other materials and fabrication methods may be suitable.

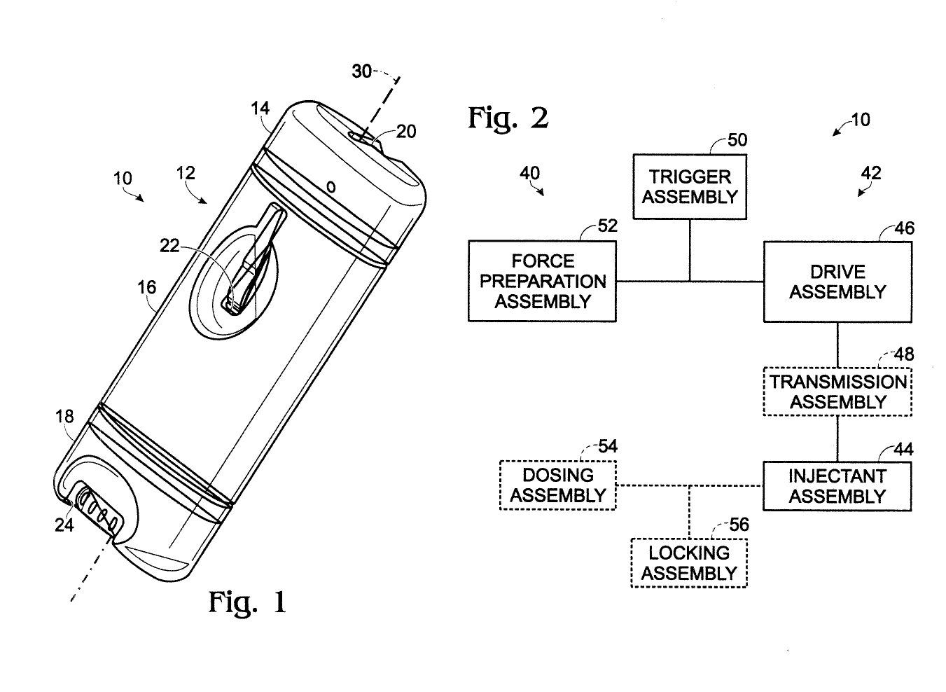

Device 10 may include one or more systems to effect an injection. For example, the device of FIG. 2 includes a priming system 40 and a delivery system 42. Priming system 40 prepares the device for delivery of an injection. Delivery system 42 provides an interface for delivery of an injectate to a recipient and delivers an injection by expelling the injectate from the device. Delivery system 42 is configured to expel a volume of fluid from the device, such as a drug. The word “drug” as used herein is intended to encompass, for example, and without limitation, any medication, pharmaceutical, therapeutic, vaccine, or other material which can be administered by injection.

Delivery system 42 includes an injectate assembly 44 for housing an injectate and providing an interface with a recipient's skin. The delivery system also includes a drive assembly 46 to provide a driving force to effect an injection. In some versions of the device, a transmission assembly 48 may be provided to couple the injectate assembly and the drive assembly. A trigger assembly 50 assists a user in selectively actuating the drive assembly, directly or indirectly via the transmission assembly, to deliver an injection.

Priming system 40 includes a force-preparation assembly 52 to selectively arrange the drive assembly to provide a drive force to deliver an injection. In some versions of the device, a dosing assembly 54 may be included to assist a user in preparing a specific dose to be injected. The priming system may include a locking assembly 56 to releasably retain injectate assembly 44 relative to body 20 and/or couple dosing assembly 54 and injectate assembly 44 in conjunction with the transmission assembly.

Device 10 may include aspects of the device described in U.S. Patent Application Publication No. 2005/0119608 A1, the disclosure of which is incorporated herein by reference, to prepare the device for delivery of an injection.

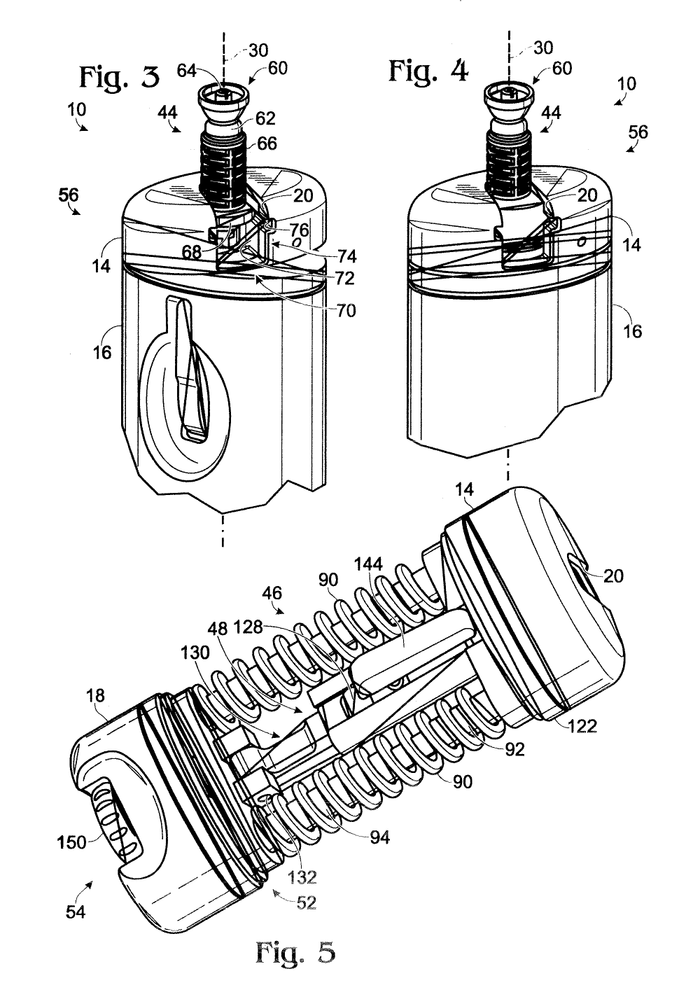

As illustrated in FIGS. 3 and 4, injectate assembly 44 includes a nozzle 60 forming a liquid chamber 62 with an outlet orifice 64. The liquid chamber may include a dose scale 66 to incrementally measure the volume of the liquid chamber. In the example shown in FIG. 3, dose scale 66 is a pre-molded dose scale having ribs to indicate each unit of measure. In some versions of the device, the dose scale includes indicia to inform a user of the volume of the liquid chamber.

Injection device 10 may be configured to be reused for multiple injections. In such a configuration, it may be desirable to periodically replace the nozzle with a fresh unused nozzle, such as to reduce contamination risks. Nozzle 60 may include one or more extensions to assist a user in locating the injectate assembly relative to the rest of the device. Injectate assembly 44 may be coupled to the device by placing the nozzle through opening 20 in the front housing, such as by sliding the nozzle laterally through the opening, as illustrated in FIG. 3. In such a configuration, the nozzle includes an extension in the form of a guide lip 68. The guide lip and opening may be similarly shaped to assist a user in aligning the injectate assembly relative to body 12.

In reusable configurations in which the injectate assembly is selectively engageable with body 12, priming system 40 includes locking assembly 56 configured to releasably couple the injectate assembly to the body. The locking assembly includes a coupling portion 70 configured to receive the injectate assembly. In the exemplary device of FIGS. 3 and 4, the coupling portion takes the form of a chamber 72 that is accessed through opening 20 in the front housing. The locking assembly may include one or more alignment portions 74 configured to locate the injectate assembly relative to the coupling portion. In some versions of the device, the alignment portion takes the form of a channel 76 configured to receive a portion of the nozzle, such as guide lip 68, and thereby align the nozzle within the locking assembly.

At least some of body 12 may be movable relative to locking assembly 56 and configured to selectively retain injectate assembly 44 within the coupling portion. For example, front housing 14 may be configured to move opening 20 relative to coupling portion 70, such as by rotating about axis 30. Front housing 14 may therefore be movable between an open position, in which the coupling portion is accessible, as shown in FIG. 3, and a closed position, in which the coupling portion is not accessible, as shown in FIG. 4. Body 12 may incorporate any other suitable method and/or mechanism to retain the injectate assembly within the coupling portion. For example, body 12 may include a flap or other movable structure that selectively covers opening 20.

Locking assembly 56 may be alterable to accommodate various injectate assemblies. For example, front housing 14 may be removable and/or exchangeable to couple injectate assemblies of various configurations and sizes to the device. Coupling portion 70 may include interchangeable components or other coupling components, such as locking pins, and the like.

In the reusable configuration described above, injectate assembly 44 may be selectively engageable with body 12. However, it should be appreciated that the injectate assembly may be permanently retained in, or coupled to, body 12 prior to providing the device to a user, such as for single-use, disposable devices as disclosed in U.S. Pat. Nos. 6,264,629 and 6,132,395, the disclosures of which are incorporated herein by reference.

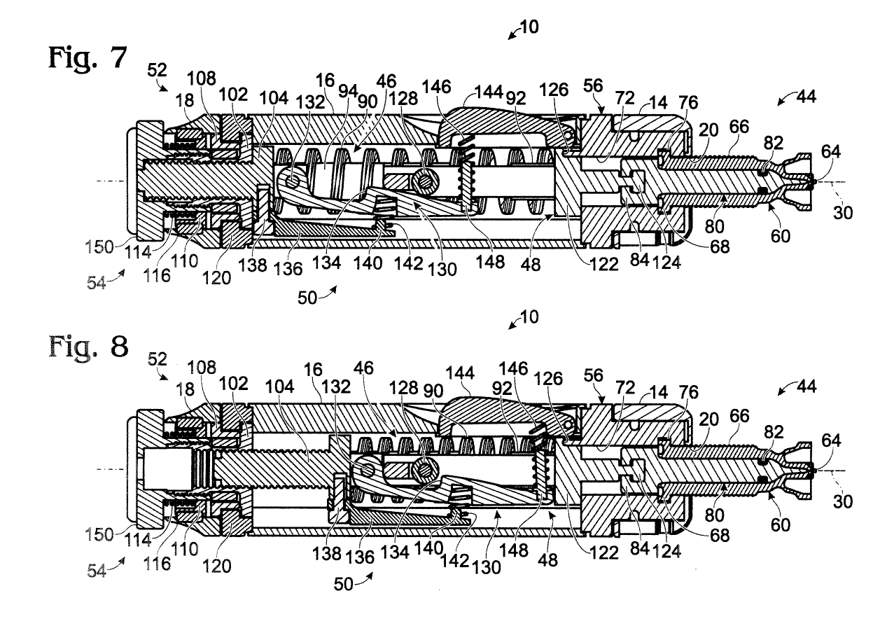

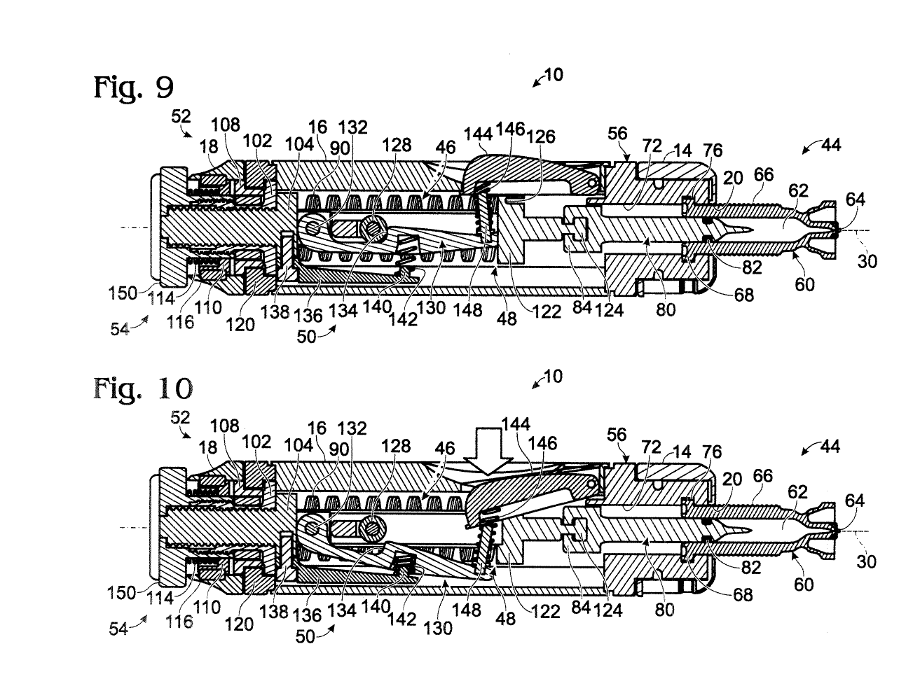

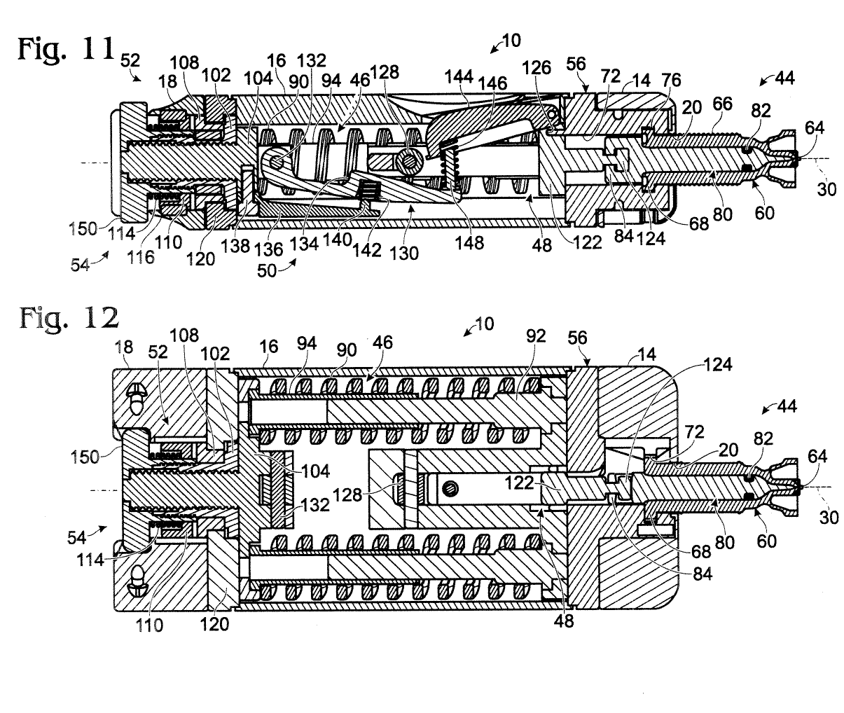

FIGS. 5-12 illustrate internal components of an exemplary injection device. The exemplary device is alterable between a plurality of configurations. For example, FIG. 7 illustrates a stored configuration in which drive assembly 46 is not operatively coupled to injectate assembly 44. FIG. 8 illustrates a primed configuration in which the drive assembly operatively couples with the injectate assembly. FIG. 9 illustrates a dosed configuration in which the injectate assembly may be filled with the fluid to be injected. FIG. 10 illustrates initiation of a fired configuration in which the drive assembly transmits a driving force to the injectate assembly. FIG. 11 illustrates the fired configuration in which the contents of the injectate assembly have been fully expelled. Upon completion of this injection sequence, the device may return to its stored configuration, as shown in FIG. 7.

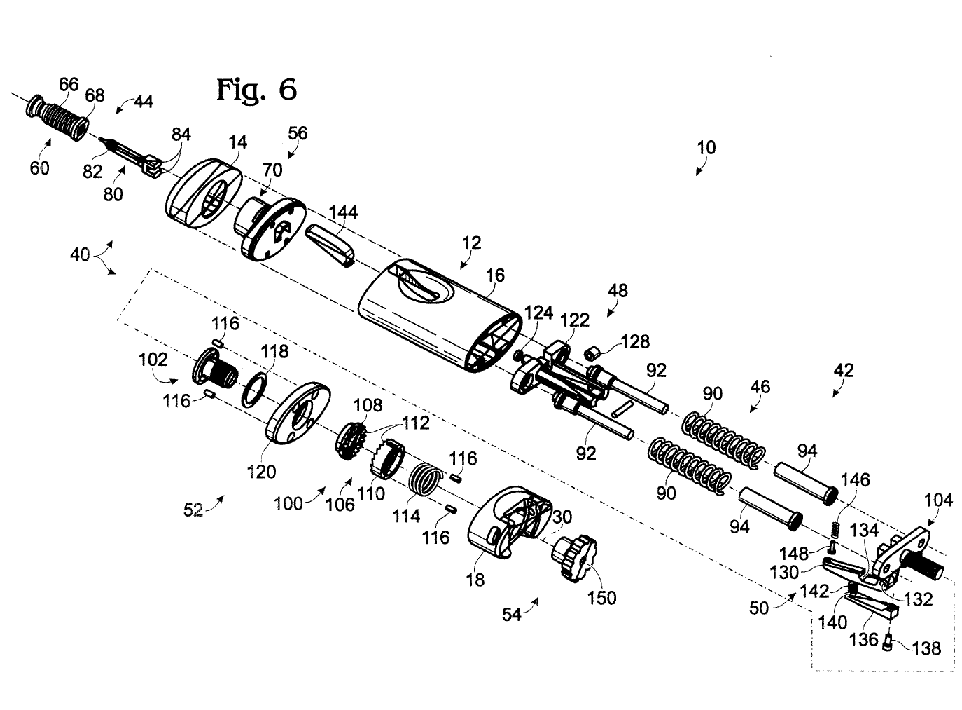

As shown in FIGS. 6-12, a plunger 80 is selectively movable within nozzle 60 and varies the volume of the liquid chamber. Plunger 80 may include a seal 82, such as an O-ring, to prevent fluid from leaking into the device. The plunger may include gripping members 84 to assist in coupling of the injectate assembly to the rest of the device and provide a means of varying the liquid chamber volume.

As previously noted, delivery system 42 includes drive assembly 46 to provide a driving force that effects an injection by expelling fluid from the injectate assembly. For example, the drive assembly may move plunger 80 within the liquid chamber. As depicted in FIGS. 7-12, gripping members 84 may operatively couple the plunger with the drive assembly. The gripping members may be coupled to the drive assembly directly or indirectly. In the exemplary device of FIGS. 6-12, the delivery system further includes a transmission assembly 48 to transmit the driving force provided by the drive assembly to the injectate assembly through coupling with the gripping members of the plunger. The gripping members may assist in aligning the injectate assembly relative to the body, as shown in FIG. 3.

The drive assembly may include one or more injection springs 90. The springs may be offset from an injection axis 30. For example, in the exemplary configuration of FIGS. 5-12, the drive assembly includes a pair of parallel springs. As shown, injection springs 90 are maintained parallel with axis 30 by suitably configured supporting rods, namely, a pair of forward rods 92 and a pair of rearward rods 94. The supporting rods are configured to move relative to one another, such as the forward rods within the rearward rods, as the compression of the springs and their subsequent length is altered. The injection springs may be maintained in a constant state of compression to maintain contact with the ends of the supporting rods. The use of two or more springs may allow for smaller springs and a greater variety of component layouts. For example, a pair of parallel springs may be configured to deliver the same drive force as a single spring while being more compact and providing more space along injection axis 30 for other components, such as the transmission and trigger assemblies. The multiple springs may be longer than a single spring and have a lower spring rate, which may provide more uniform power and maintain a more consistent pressure than traditional single spring designs.

As shown in FIG. 2, priming system 40 includes force-preparation assembly 52 that prepares the delivery system, namely, the drive assembly, to provide a driving force to the injectate assembly. For example, the force-preparation assembly may prepare the device to be powered by pressurized gas, one or more springs, an electric motor, a pyrotechnic charge, or any other suitable source of power.

In the exemplary device of FIGS. 5-12, force-preparation assembly 52 is configured to compress a pair of injection springs 90. The force-preparation assembly may include a winder 100 and a winder compressor 102. The winder compressor communicates with a rearward spring stop or spring compressor 104. The winder is configured to selectively urge the spring compressor towards the pair of springs via rotation of the winder compressor. Forward supporting rods 92 slide relative to rearward supporting rods 94, thereby adjusting the overall length of the rods as the springs are compressed.

The winder may include a clutch mechanism 106 to prevent excessive compression of the springs. For example, the clutch mechanism may take the form of a first winder portion 108 and a second winder portion 110 that may be configured to selectively disengage from each other to prevent further movement of the winder compressor. As most clearly shown in the exploded view of FIG. 6, the winder portions include protrusions 112, such as ratchet teeth, to engage one another. The winder may be biased to engage the winder compressor, such as by a spring 114 which urges second winder portion 110 towards first winder portion 108.

The force-preparation assembly components may be coupled to one another or to body 12 using one or more pins 116. The force-preparation assembly may include any suitable components to assist in relative movement of the assembly or coupling of components, including, but not limited to, bushing 118 and plate 120.

As previously noted, transmission assembly 48 operatively couples the drive assembly with the injectate assembly. In the exemplary device of FIGS. 6-12, the transmission assembly includes a forward spring stop or ram 122 to deliver the driving force of injection springs 90 to plunger 80. The ram is coupled to forward supporting rods 92 and includes an extension 124 that is coupled to gripping members 84 of the plunger. Constant spring compression may bias the extension to a position suitable to receive the injectate assembly, such as by extending into the chamber of the locking assembly to engage with the gripping members upon insertion of the injectate assembly into the locking assembly, as shown in FIG. 3.

Transmission assembly 48 may be configured to cooperate with body 12 to ensure appropriate alignment of the device components. For example, the exemplary device shown in FIGS. 7-12 includes an alignment recess 126 extending from ram 122 to engage an interior portion of the body.

As illustrated in FIGS. 6-11, the transmission assembly includes an assembly coupler 128 configured to couple the transmission assembly with trigger assembly 50. The assembly coupler may take any suitable form, such as a movable component, rotatable component, fixed protrusion or detent, and the like, with the exemplary form being a wheel or roller.

Trigger assembly 50 is configured to alter the device between at least some of the plurality of configurations. For example, the trigger assembly may assist in altering the device from a stored configuration to a primed configuration. Once the injectate assembly has been filled, the trigger assembly may alter the device to the fired configuration to deliver an injection.

The trigger assembly may include a trigger 130, such as the arm shown in FIGS. 6-11. In the example shown, trigger 130 pivots about a pin 132 and includes an arcuate latch 134 configured to engage with roller 128. As shown in FIGS. 7-9, as the springs are compressed, the trigger rotates to engage the trigger latch with the roller, thereby coupling movement of spring compressor 104 with ram 122. A tension bar 136 is coupled to the spring compressor by screw 138 and includes a protrusion 140. Resting against protrusion 140 is a spring 142 to bias the trigger to engage the roller. Once the trigger has engaged the roller, the drive assembly, transmission assembly, and trigger assembly are configured to move as a unit.

The trigger assembly includes an injection button 144 and button spring 146 for actuation of delivery of an injection. Injection button 144 is shown to be stepped in FIGS. 1, 3 and 17, and smooth in the remaining Figs. Either configuration will perform the functions required of a trigger. The button spring may be adjusted to provide suitable sensitivity of the trigger assembly and may be supported by a post 148. Once the device has been primed and dosed, a user holds the device against a recipient's skin and depresses the trigger button. As shown in FIG. 10, the trigger latch rotates away from the roller thereby releasing the ram to be propelled by the springs and against the plunger to deliver an injection.

In some versions of the device, priming system 40 includes a dosing assembly 54 that urges fluid through outlet orifice 64 into liquid chamber 62 to prepare the device to deliver a particular amount of injectate. The device may be configured to draw in a predetermined amount of injectate or an amount specified by a user. As illustrated in FIG. 9, the dosing assembly is configured to selectively urge the plunger away from the outlet orifice to increase the volume of the liquid chamber. Prior to retraction of plunger 80, nozzle 60 may be coupled with a vial, bottle, or other external supply of injectable fluid, such that, upon retraction of the plunger, a dose of injectable fluid is drawn into the liquid chamber.

Dosing assembly 54 may include a user input device of any suitable form. In the exemplary configuration of FIGS. 6-12, a dose knob 150 is provided that may be rotated to maneuver plunger 80 to intake a desired amount of injectate. The dose knob is threadably coupled to the winder compressor to rotate the winder compressor in the opposite direction as during priming and urge the spring compressor towards the rear of the device. The trigger couples movement of the ram to the spring compressor so that the interior of the device moves as a unit to withdraw the plunger relative to the nozzle.

Operation of an exemplary injection device is depicted in FIGS. 7-11. The rear housing 18 of FIG. 7 is rotated to urge spring compressor 104 to compress springs 90. Trigger 130 is biased to engage coupler 128 to maintain the springs in a compressed state, as shown in FIG. 8. Dose knob 150 is rotated to urge the coupled trigger assembly, transmission assembly, and drive assembly towards the rear of the device, thereby retracting plunger 80, as illustrated in FIG. 9. The device may then be used to deliver an injection by pressing button 144, as shown in FIG. 10.

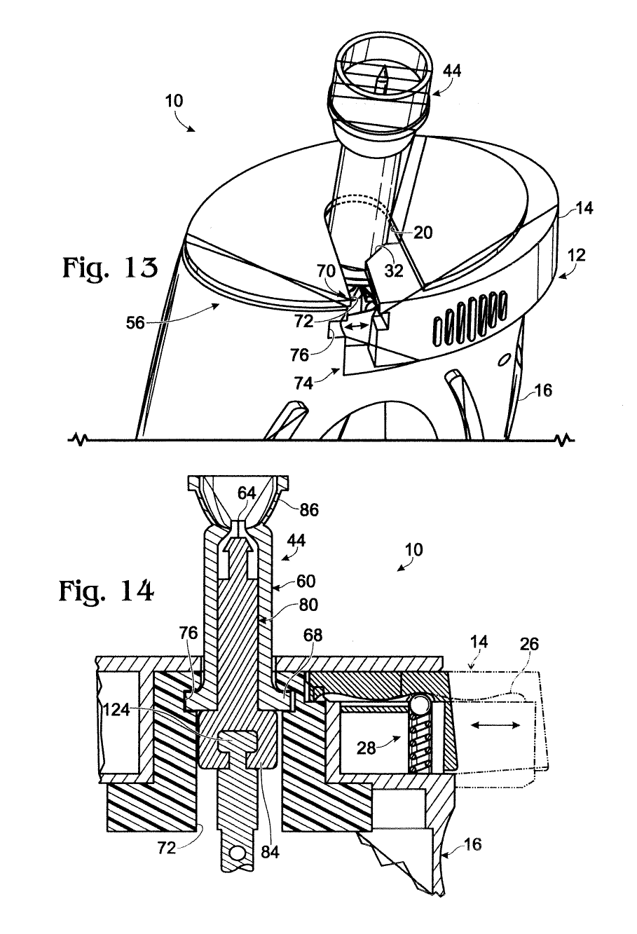

FIGS. 13-16 illustrate another exemplary locking assembly 56. As illustrated in FIG. 13, body 12 includes a front housing 14, at least a portion of which is configured to move relative to a central housing 16. For example, front housing 14, or a portion thereof, may be configured to slide relative to the central housing to provide access to a coupling portion 70 through opening 20. The locking assembly may include one or more alignment portions 74 configured to locate the injectate assembly relative to the coupling portion. For example, the alignment portion may take the form of channels 76 configured to receive a portion of the injectate assembly, such as guide lip 68 of the nozzle assembly, and thereby align the nozzle within the locking assembly. As the injectate assembly is inserted into the locking assembly, gripping members 84 of plunger 80 operatively couple the plunger with the drive assembly.

As shown in FIG. 14, at least a portion of front housing 14 is movable relative to the central housing. The slidable portion of the front housing may be biased to maintain one or more positions. For example, the front housing may be biased to move to an open position and/or a closed position once it has moved a predetermined amount. As shown, the device includes a notched track 26 and a biasing mechanism 28. The biasing mechanism engages the track to move between the notched positions. For example, the track may include a notch that corresponds to an open position and a notch that corresponds to a closed position of the locking assembly. The device is thereby configured to assist a user in altering the locking assembly between the open and closed positions. The biasing mechanism may include a spring-biased bearing or other suitable structure.

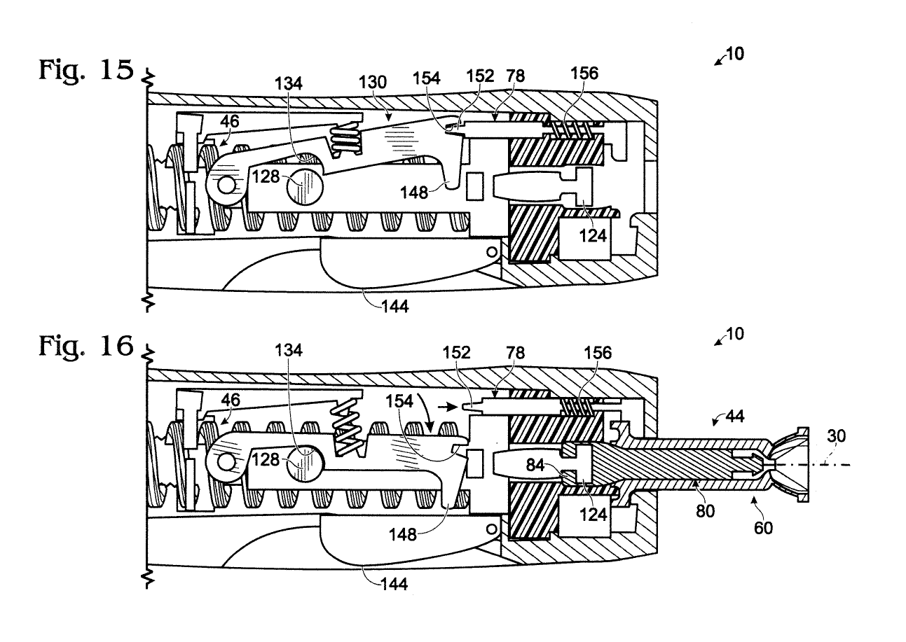

FIGS. 15 and 16 depict cross-sectional views of the locking assembly of FIGS. 13 and 14 without an injectate assembly and with an injectate assembly inserted. The locking assembly may include a latch restriction mechanism 78 that restricts coupling of trigger 130 with assembly coupler 128. Consequently, the drive assembly may not be prepared to deliver an injection until an injectate assembly is properly inserted into the device. For example, injection springs 90 cannot be maintained in a compressed state until trigger latch 134 engages the roller. An exemplary latch restriction mechanism is shown in FIGS. 15 and 16 and includes a protrusion 152 configured to engage a groove 154 in the trigger. The latch restriction mechanism may be biased, such as by a spring 156, to urge the protrusion to engage the groove to restrict movement of the trigger towards assembly coupler 128. As shown in FIG. 15, when an injectate assembly is not inserted in the locking assembly, the locking assembly engages the trigger to restrict rotation of the trigger. When an injectate assembly is properly installed, as shown in FIG. 16, the latch restriction mechanism is moved away from the trigger so that the trigger is free to rotate and engage the assembly coupler 128. This mechanism may therefore prevent inadvertent or mistaken firing of the device without the injectate assembly in place, which can otherwise lead to damage of the device.

The locking assembly of FIGS. 13-16 may also be biased to urge the injectate assembly out of the locking assembly. For example, spring 156 may urge the latch restriction mechanism towards the left, as shown in FIGS. 15 and 16, to urge the injectate assembly out of chamber 72, such as to assist a user in replacing the injectate assembly. As shown in FIG. 13, the front housing may include a chamfered edge 32 to urge the injectate assembly into the chamber to assist a user in inserting a new injectate assembly.

Also shown in FIGS. 13, 14, and 16 is a nozzle 60 including an optional section 86 to locate the injection orifice 64 a distance away from the recipient's skin, such as an intradermal spacer. Alternatively, or additionally, this section may be used as a fitting for a vial adaptor. The nozzle may include further include any of the previously discussed aspects as are suitable, such as the dose scale 66 shown in FIG. 3.

In some versions of device 10, the device may provide feedback or instructions to a user of the device. For example, the device may include one or more apertures or windows to provide a user with access to device controls or configuration status. The device may allow a user to view indicia or interior components through the body. For example, indicia, such as arrows or text, may instruct a user in proper operation of the device or convey information to a user, such as whether the device is in the stored or primed configuration.

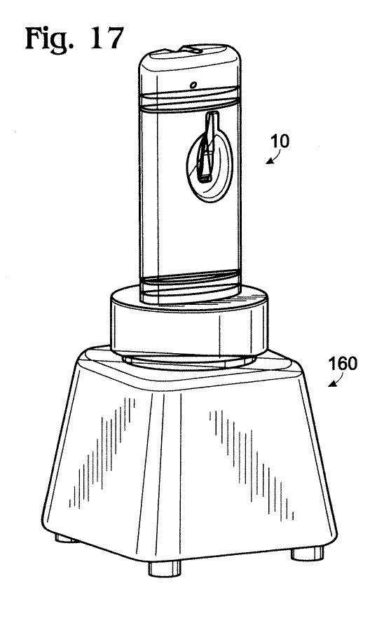

FIG. 17 illustrates an exemplary electric winding device 160 suitable for use with the above-described injection device 10. The electric winding device is configured to selectively actuate the preparation assembly. For example, device 160 may rotate rear housing 26 relative to central housing 24 to compress the injection springs. A user may hold central housing 24 to urge the injection device towards the winding device to automatically engage the winding device to rotate the rear housing. The winding device may include any suitable drive assembly, such as an electric motor, and any suitable sensors to actuate the winding device upon engagement with an injection device.

As previously described, dosing assembly 54 may include a user input device of any suitable form. In the exemplary configuration of FIGS. 6-12, dose knob 150 is provided to maneuver plunger 80 to intake a desired amount of injectate. However, it may also be possible to delete dose knob 150 and instead have winder 18 be used to both pre-load the spring for firing, and load the desired amount of injectate into nozzle 60. Specifically, the rear housing 18 depicted in FIG. 7 would still be included, and would be rotated in a first direction to urge spring compressor 104 to compress springs 90. Trigger 130 would still be biased to engage coupler 128 to maintain the springs in a compressed state, as shown in FIG. 8. However, instead of dose knob 150 being rotated to urge the coupled trigger assembly, transmission assembly, and drive assembly toward the rear of the device to retract plunger 80 as illustrated in FIG. 9, dose knob 150 may be deleted so that winder 18 would simply be rotated in a second, opposite direction to load injectate into nozzle 60. To facilitate this operation, spring 114 would be deleted. First winder portion 108 and a second winder portion 110 would remain as depicted, as would ratchet teeth 112 and pins 116. In place of dose knob 150, an inset member (not shown) would be provided to fulfill the same function as dose knob 150, but would not be accessible by the operator. This member would be axially stationary and would thread onto winder compressor 102 in the same fashion as dose knob 150 in the previously-described embodiment. This would permit first and second winder portions 108 and 110 to turn together as rear housing 18 is rotated in the second direction to draw injectate into the nozzle. Thus, while dose knob 150 would not be included in this embodiment, the operation provided by it, and most of the components that extend from it, remain, and perform the same function as if the dose knob was included. It has been determined that for certain applications, and for certain types of patients, the deletion of the dose knob renders the unit easier to operate.

Although the present device has been shown and described with reference to the foregoing operational principles and preferred embodiments, it will be apparent to those skilled in the art that various changes in form and detail can be made without departing from the spirit and scope of the invention. The present invention is intended to embrace all such alternatives, modifications and variances. The subject matter of the present invention includes all novel and non-obvious combinations and subcombinations of the various elements, features, functions and/or properties disclosed herein. Inventions embodied in various combinations and subcombinations of features, functions, elements, and/or properties may be claimed through presentation of claims in a subsequent application.

What is claimed is:

1. A needle-free injection device comprising:
a body configured to house a plurality of systems;
a delivery system comprising an injectate assembly including a nozzle forming a liquid chamber with an outlet orifice and a plunger selectively movable within the nozzle, the delivery system being adapted to expel a volume of liquid from the liquid chamber; and
a priming system adapted to prepare the device for delivery of an injection, the priming system comprising:
a locking assembly adapted to releasably retain the injectate assembly relative to the body, the locking assembly including a coupling portion configured to receive the injectate assembly and an alignment portion configured to locate the injectate assembly relative to the coupling portion, wherein at least some of the body is movable relative to the locking assembly and configured to selectively retain the injectate assembly within the coupling portion.

2. The device of claim 1, wherein the alignment portion includes one or more channels adapted to receive a portion of the nozzle.

3. The device of claim 1, wherein the body includes a first housing and a second housing, the first housing configured to rotate relative to the second housing and alter the locking assembly between an open position, in which the coupling portion is accessible, and a closed position, in which the coupling portion is not accessible.

4. The device of claim 1, wherein the body includes a first housing and a second housing, the first housing configured to slide relative to the second housing and alter the locking assembly between an open position, in which the coupling portion is accessible, and a closed position, in which the coupling portion is not accessible.

5. The device of claim 1, further comprising a priming system adapted to prepare the device for delivery of an injection, wherein the locking assembly is configured to restrict actuation of the priming system.

6. The device of claim 1, wherein the priming system further comprises a dosing assembly adapted to selectively urge the plunger away from the outlet orifice to increase volume of the liquid chamber.

7. The device of claim 6, wherein the nozzle includes a dose scale configured to incrementally measure the volume of the liquid chamber.

8. The device of claim 5, wherein the locking assembly includes a latch restriction mechanism for preventing actuation of the priming system when the nozzle is not in place.

9. The device of claim 8, further comprising:
a drive assembly configured to deliver an operative force to the plunger;
a transmission assembly displaceable along an axis and adapted to couple the injectate assembly and the drive assembly, the transmission assembly including an assembly coupler; and
a trigger assembly including a trigger adapted to selectively engage the assembly coupler and move the drive assembly and the plunger as a unit;
wherein the latch restriction mechanism prevents engagement of the trigger with the transmission assembly.

10. The device of claim 9, wherein the latch restriction mechanism includes a protrusion configured to engage a groove in the trigger.

11. The device of claim 10, wherein the latch restriction mechanism is biased to urge the protrusion to engage the groove to restrict movement of the trigger towards the assembly coupler.

12. The device of claim 11, wherein upon the injectate assembly being installed within the coupling portion, the protrusion is moved away from the groove so that the trigger is free to engage the assembly coupler.

13. The device of claim 11, wherein the locking assembly is biased to urge the injectate assembly out of the locking assembly.

14. The device of claim 13, wherein the latch restriction mechanism includes a spring that is configured to urge the latch restriction mechanism towards the groove, thereby urging the injectate assembly out of the coupling portion.

15. The device of claim 3, wherein the first housing includes a chamfered edge to urge the injectate assembly into the coupling portion.

16. The device of claim 4, wherein the first housing includes a chamfered edge to urge the injectate assembly into the coupling portion.
